(19) United States
(12) Patent Application Publication
(10) Pub. No.: US 2025/0228564 A1
(43) Pub. Date: Jul. 17, 2025

(54) Suturing Device

(71) Applicants: Shang-Chih LIN, Taipei City (TW); Anasto Medical Inc., Taipei City (TW)

(72) Inventors: Shang-Chih LIN, Taipei City (TW); Irene HL HUANG, Taipei City (TW); Eric Lynn HUANG, Taipei City (TW)

(73) Assignees: Shang-Chih LIN, Taipei City (TW); Anasto Medical Inc., Taipei City (TW)

(21) Appl. No.: 18/412,665

(22) Filed: Jan. 15, 2024

(51) Int. Cl. A61B 17/115; A61B 17/00; A61B 90/00

(52) U.S. Cl. CPC A61B 17/115; A61B 90/06; A61B 2017/00017; A61B 2017/00384; A61B 2017/00982

(57) ABSTRACT

A suturing device is adapted for sewing two tubular objects together, and includes a suturing aid rod, a plurality of sewing subunits, and two pushing subunits. The suturing aid rod has two carrying portions that are opposite to each other in a first direction. Each of the sewing subunits includes a suture thread, and two suture needles that are respectively connected to two opposite ends of the suture thread. Each of the pushing subunits is slidable relative to a respective one of the carrying portions between an initial position and a puncture position. When the pushing subunits are in the puncture position, one of the suture needles of each of the sewing subunits is adapted to puncture one of the tubular objects, and the other one of the suture needles of each of the sewing subunits is adapted to puncture the other one of the tubular objects.

### FIELD

The disclosure relates to an aid, and more particularly to an aid to suturing.

### BACKGROUND

A conventional surgical anastomosis for connecting two tubular body structures (e.g., blood vessels and lymphatic vessels) is a common surgical technique. However, the conventional surgical anastomosis may be time-consuming and difficult to perform because the tubular body structures are generally small in size and have to be sewn circumferentially.

### SUMMARY

Therefore, an object of the disclosure is to provide a suturing device that can alleviate at least one of the drawbacks of the prior art.

According to the disclosure, the suturing device is adapted for sewing two tubular objects together. Each of the tubular objects has a connecting opening. The suturing device includes a suturing unit that includes a suturing aid rod, a plurality of sewing subunits, and two pushing subunits. The suturing aid rod is elongated in a first direction, and has two carrying portions that are opposite to each other in the first direction. Each of the carrying portions has a carrying section that is proximate to another one of the carrying portions, an insertion section that is distal from the another one of the carrying portions and that is adapted to be inserted into the connecting opening of a respective one of the tubular object, and a plurality of grooves that are disposed about an axis extending in the first direction. For each of the carrying portions, each of the grooves extends in the first direction, and has a first accommodating part that is located at the insertion section, and that has a curved area and a needle opening spatially communicating with the curved area and located at the insertion section. The curved area of the first accommodating part of each of the grooves has two side segments that are spaced apart from each other in the first direction and that are distal from the axis, and a middle segment that interconnects the side segments and that is proximate to the axis. Each of the sewing subunits includes a suture thread, and two suture needles that are respectively connected to two opposite ends of the suture thread. One of the suture needles of each of the sewing subunits is disposed on the curved area of the first accommodating part of a respective one of the grooves of one of the carrying portions, and has a cross-section that is shaped to correspond to a cross-section of the curved area of the first accommodating part of the respective one of the grooves of the one of the carrying portions. The other one of the suture needles of each of the sewing subunits is disposed on the curved area of the first accommodating part of a respective one of the grooves of the other one of the carrying portions and has a cross-section that is shaped to correspond to a cross-section of the curved area of the first accommodating part of the respective one of the grooves of the other one of the carrying portions. Each of the pushing subunits is mounted to a respective one of the carrying portions, and is slidable relative to the respective one of the carrying portions between an initial position and a puncture position in the first direction. The pushing subunits are proximate to a center of the suturing aid rod when the pushing subunits are in the initial position. The pushing subunits are operable to move away from each other toward the puncture position so that the pushing subunits respectively push the suture needles of the sewing subunits and urge the suture needles to respectively slide along the curved areas of the grooves and respectively protrude out of the needle opening of the grooves. When the pushing subunits are in the puncture position, one of the suture needles of each of the sewing subunits is adapted to puncture one of the tubular objects, and the other one of the suture needles of each of the sewing subunits is adapted to puncture the other one of the tubular objects.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a perspective view of a first embodiment of a suturing device according to the disclosure.

FIG. 2 is another perspective view of the first embodiment illustrating a suturing unit of the first embodiment being released from an actuation unit of the first embodiment.

FIG. 3 is a perspective view of the suturing unit.

FIG. 4 is a partly exploded perspective view of the suturing unit.

FIG. 5 is a fragmentary sectional view of the first embodiment illustrating pushing subunits of the first embodiment in an initial position and the actuation subunit of the first embodiment in an initial state.

FIG. 6 is a fragmentary sectional view of a suture needle and a groove of the first embodiment.

FIG. 7 is a fragmentary sectional view of the suture needle and a modification of the groove.

FIG. 8 is a sectional view of the first embodiment illustrating the pushing subunits in the initial position and the actuation subunit in the initial state.

FIG. 9 is another sectional view of the first embodiment.

FIG. 10 is a partly exploded perspective view of the actuation unit.

FIG. 11 is a view similar to FIG. 5 but illustrating the pushing subunits in a puncture position and the actuation subunit in an actuation state.

FIG. 12 is a sectional view of the first embodiment illustrating the pushing subunits in the puncture position and the actuation subunit in the actuation state.

FIG. 13 is a fragmentary sectional view illustrating the pushing subunits being respectively pushed by pushing surfaces of the first embodiment when in the puncture position.

FIG. 14 is a schematic view of tubular objects that may be sewn by the first embodiment.

FIG. 15 is a schematic view of other tubular objects that may be sewn by the first embodiment.

FIG. 16 is a partly exploded perspective view of a suturing unit of a second embodiment of the suturing device according to the disclosure.

FIG. 17 is a perspective view of part of an actuation unit of the second embodiment.

FIG. 18 is a perspective view of part of a third embodiment of the suturing device according to the disclosure.

FIG. 19 is a perspective view of an actuation unit of a fourth embodiment of the suturing device according to the disclosure.

FIG. 20 is a perspective view of a fifth embodiment of the suturing device according to the disclosure.

FIG. 21 is another perspective view of the fifth embodiment illustrating a suturing unit of the fifth embodiment being released from an actuation unit of the fifth embodiment.

FIG. 22 is a partly exploded perspective view of the suturing unit of the fifth embodiment.

FIG. 23 is a fragmentary sectional view of the fifth embodiment illustrating pushing subunits of the fifth embodiment in an initial position and the actuation subunit of the fifth embodiment in an initial state.

FIG. 24 is a view similar to FIG. 23 but illustrating the pushing subunits in a puncture position and the actuation subunit in an actuation state.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

It should be noted herein that for clarity of description, spatially relative terms such as “top,” “bottom,” “upper,” “lower,” “on,” “above,” “over,” “downwardly,” “upwardly” and the like may be used throughout the disclosure while making reference to the features as illustrated in the drawings. The features may be oriented differently (e.g., rotated 90 degrees or at other orientations) and the spatially relative terms used herein may be interpreted accordingly.

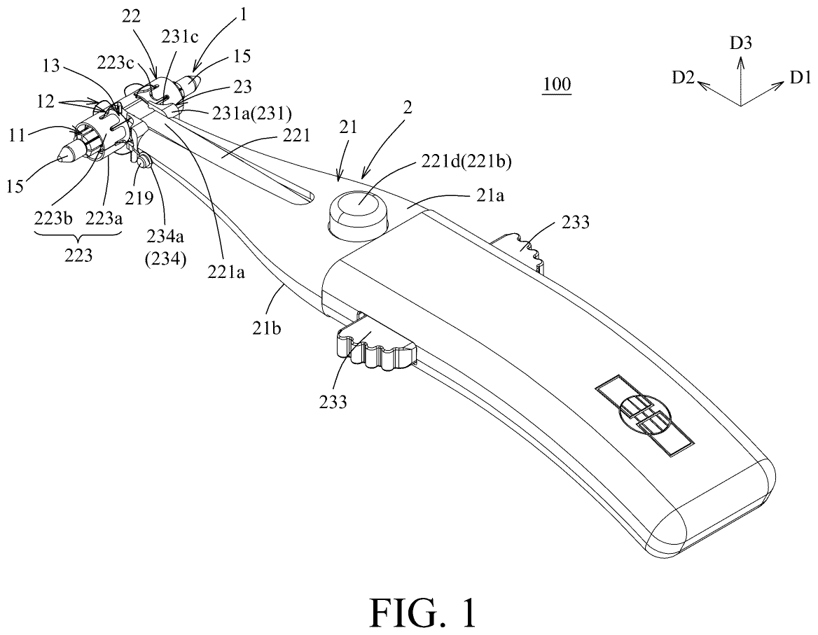

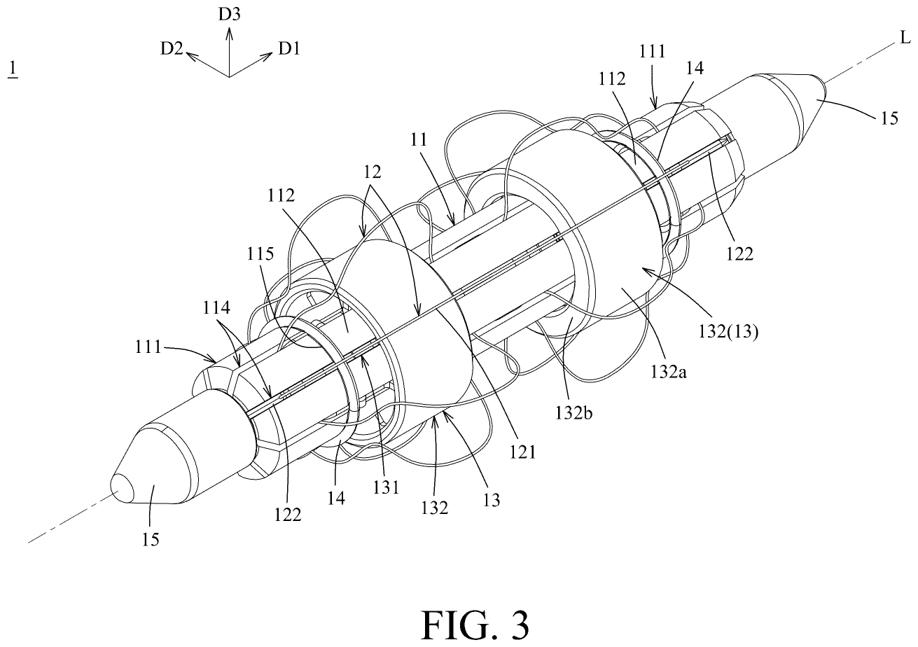

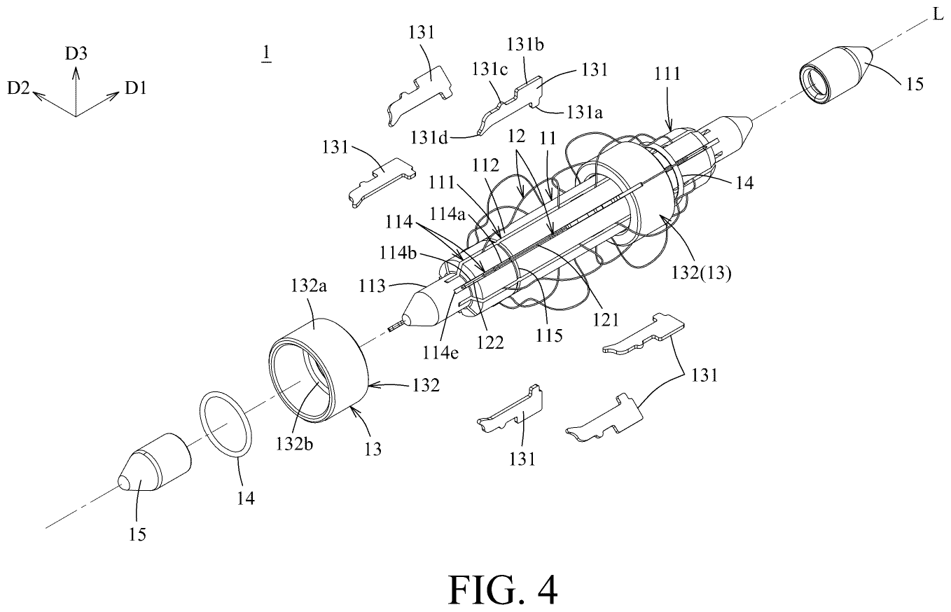

Referring to FIGS. 1 to 5, a first embodiment of a suturing device 100 according to the disclosure is adapted for sewing two tubular objects 200 together. Each of the tubular objects 200 may be a tubular body structure (e.g., a blood vessel or a lymphatic vessel) that ruptures, and has a connecting opening 201. The suturing device 100 includes a suturing unit 1 and an actuation unit 2.

The suturing unit 1 includes a suturing aid rod 11, a plurality of sewing subunits 12, two pushing subunits 13, two annular resilient members 14, and two groove covers 15. The suturing aid rod 11 may be, but not limited to be, made of a metal material. The suturing aid rod 11 is elongated in a first direction (D1), and has two carrying portions 111 that are opposite to each other in the first direction (D1). Each of the carrying portions 111 has a carrying section 112 that is proximate to another one of the carrying portions 111, an insertion section 113 that is distal from the another one of the carrying portions 111 (i.e., the carrying sections 112 of the carrying portions 111 are located between the insertion sections 113 of the carrying portions 111 in the first direction (D1)) and that is adapted to be inserted into the connecting opening 201 of a respective one of the tubular objects 200, a plurality of grooves 114 that are equiangularly disposed about an axis (L) extending in the first direction (D1), and an annular indentation section 115 that is located at the carrying section 112 and that extends circumferentially. For each of the carrying portions 111, a diameter of the insertion section 113 may be smaller than that of the carrying section 112, and the insertion section 113 may taper away from the carrying section 112 so that the insertion section 113 may be relatively easy to be inserted into the connecting opening 201 of the respective one of the tubular objects 200.

For each of the carrying portions 111, each of the grooves 114 extends in the first direction (D1), and has a first accommodating part 114b and a second accommodating part 114a. For each of the carrying portions 111, the second accommodating part 114a of each of the grooves 114 is located at the carrying section 112 and has an indented area 114c, and the first accommodating part 114b of each of the grooves 114 extends from the insertion section 113 to the carrying section 112, and has a curved area 114d and a needle opening 114e that spatially communicates with the curved area 114d and that is located at the insertion section 113. The curved area 114d of the first accommodating part 114b of each of the grooves 114 has two side segments that are spaced apart from each other in the first direction (D1) and that are distal from the axis (L), and a middle segment that interconnects the side segments and that is proximate to the axis (L).

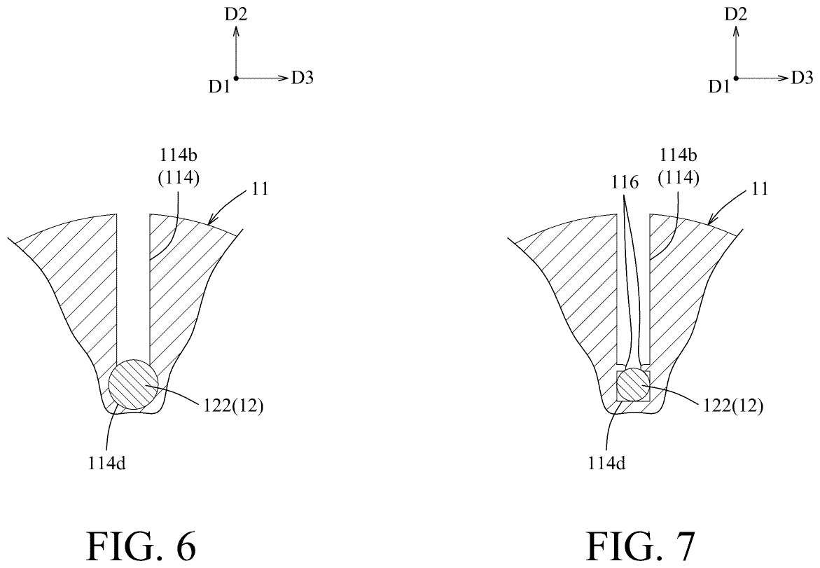

Each of the sewing subunits 12 includes a suture thread 121 and two suture needles 122 that are respectively connected to two opposite ends of the suture thread 121. One of the suture needles 122 of each of the sewing subunits 12 is disposed on the curved area 114d of the first accommodating part 114b of a respective one of the grooves 114 of one of the carrying portions 111, and has a cross-section that is shaped to correspond to a cross-section of the curved area 114d of the first accommodating part 114b of the respective one of the grooves 114 of the one of the carrying portions 111. The other one of the suture needles 122 of each of the sewing subunits 12 is disposed on the curved area 114d of the first accommodating part 114b of a respective one of the grooves 114 of the other one of the carrying portions 111, and has a cross-section that is shaped to correspond to a cross-section of the curved area 114d of the first accommodating part 114b of the respective one of the grooves 114 of the other one of the carrying portions 111. It is noted that, referring further to FIG. 6, in the first embodiment, the cross section of each of the suture needles 122 of the sewing subunits 12 may be shaped to correspond to the cross section of the curved area 114d of the first accommodating part 114b of the respective one of the grooves 114 of the carrying portions 111 (in this embodiment, each of the grooves 114 widens at the curved area 114d thereof). However, referring further to FIG. 7, in a modification of the first embodiment, each of the grooves 114 of the carrying portions 111 may further have two needle-refraining protrusion parts 116 that are adjacent to the curved area 114d of the groove 114 and that cooperate with the curved area 114d of the groove 114 to refrain the respective one of the suture needles 122 of the sewing subunits 12 from leaving the curved area 114d of the groove 114 in a radial direction of the suturing aid rod 11. In another modification of the first embodiment, the suture needles 122 may be refrained from leaving the curved areas 114d of the grooves 114 in the radial direction of the suturing aid rod 11 by any configuration of each of the curved areas 114d of the grooves 114 other than those mentioned in the above examples.

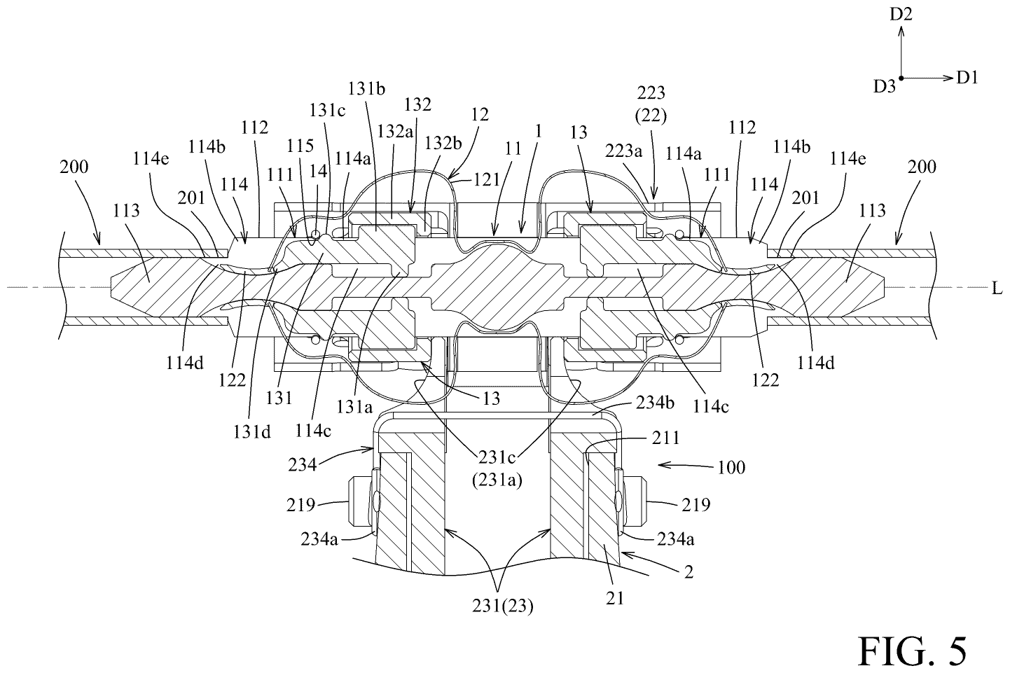

Each of the pushing subunits 13 is mounted to a respective one of the carrying portions 111, and is slidable relative to the respective one of the carrying portions 111 between an initial position (see FIG. 5) and a puncture position (see FIG. 11) in the first direction (D1). Each of the pushing subunits 13 includes a plurality of sliding plates 131 that are respectively disposed in the second accommodating parts 114a of the grooves 114 of the respective one of the carrying portions 111, and a pushing ring 132 that is slidably mounted to the respective one of the carrying portions 111 and that is operable to push the sliding plates 131. Each of the sliding plates 131 and the pushing ring 132 of the pushing subunits 13 may be, but not limited to be, made of a metal material.

The pushing subunits 13 are proximate to a center of the suturing aid rod 11 when the pushing subunits 13 are in the initial position. The pushing rings 132 of the pushing subunits 13 are operable to move away from each other toward the puncture position so that the pushing ring 132 of each of the pushing subunits 13 pushes the sliding plates 131 of the pushing subunit 13 toward the first accommodating parts 114b of the grooves 114 of the respective one of the carrying portions 111, and that the sliding plates 131 of the pushing subunits 13 respectively push the suture needles 122 of the sewing subunits 12 and urge the suture needles 122 to respectively slide along the curved areas 114d of the grooves 114 and respectively protrude from the needle openings 114e of the grooves 114. When the pushing subunits 13 are in the puncture position, one of the suture needles 122 of each of the sewing subunits 12 is adapted to puncture one of the tubular objects 200, and the other one of the suture needles 122 of each of the sewing subunits 12 is adapted to puncture the other one of the tubular objects 200.

Each of the sliding plates 131 has a sliding section 131a that extends into the indented area 114c of the respective one of the grooves 114 and that is slidable relative to the indented area 114c of the respective one of the grooves 114 in the first direction (D1), a ring-surrounded section 131b that protrudes out of the second accommodating part 114a of the respective one of the grooves 114, a blocking protrusion section 131c that protrudes away from the axis (L), and a needle-pushing section 131d that extends into the first accommodating part 114b of the respective one of the grooves 114. The needle-pushing section 131d of each of the sliding plates 131 of one of the pushing subunits 13 is operable to push one of the suture needles 122 of each of the sewing subunits 12. The needle-pushing section 131d of each of the sliding plates 131 of the other one of the pushing subunits 13 is operable to push the other one of the suture needles 122 of each of the sewing subunits 12. The pushing ring 132 of each of the pushing subunits 13 has a surrounding section 132a that surrounds the ring-surrounded sections 131b of the sliding plates 131 of the pushing subunit 13, and a plate-pushing section 132b that is connected to the surrounding section 132a and that is operable to push the ring-surrounded sections 131b of the sliding plates 131 of the pushing subunit 13 away from the center of the suturing aid rod 11. By virtue of the pushing ring 132 of each of the pushing subunits 13 surrounding the sliding plates 131 of the pushing subunit 13, the sliding plates 131 of each of the pushing subunits 13 are refrained from leaving the second accommodating parts 114a of the grooves 114 of the respective one of the carrying portions 111.

The annular indentation section 115 of each of the carrying portions 111 intersects the second accommodating parts 114a of the grooves 114 of the carrying portion 111. The annular resilient members 14 are respectively disposed on the annular indentation sections 115 of the carrying portions 111. The blocking protrusion section 131c of each of the sliding plates 131 is located between the annular resilient members 14 in the first direction (D1) so that the sliding plates 131 are prevented from moving away from the center of the suturing aid rod 11 in the first direction (D1) when no external force is exerted thereon.

The groove covers 15 respectively surround the insertion sections 113 of the suturing aid rod 11. Each of the groove covers 15 covers the needle openings 114e of the grooves 114 of a respective one of the carrying portions 111. By virtue of the groove covers 15 covering the needle openings 114e, the suture needles 122 of the sewing subunits 12 are prevented from leaving the grooves 114 of the carrying portions 111 through the needle openings 114e of the grooves 114.

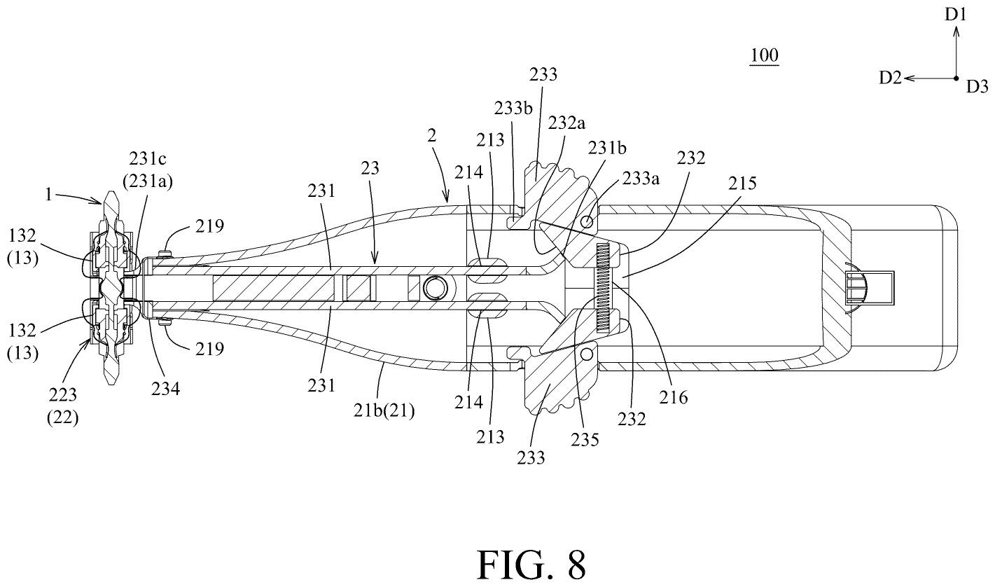

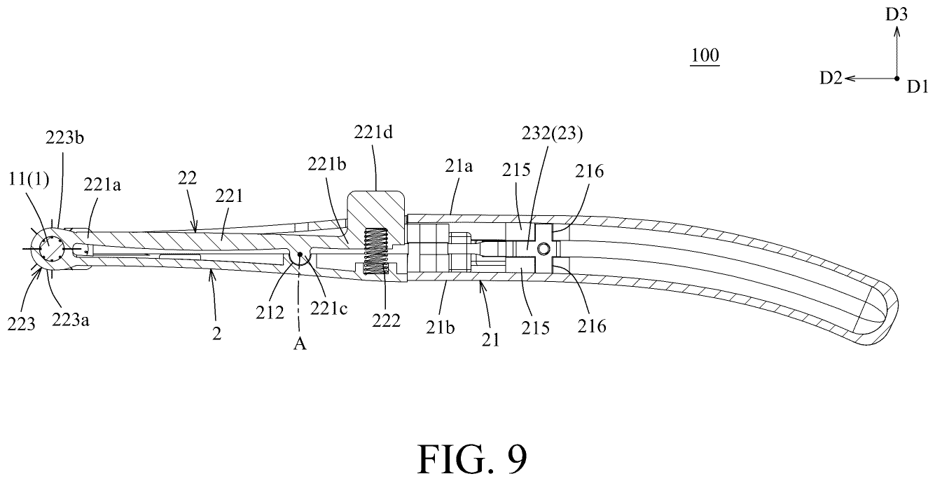

Referring further to FIGS. 8 to 10, in cooperation with FIG. 1, the actuation unit 2 is used for actuating the suturing unit 1, is substantially elongated in a front-rear direction (D2) orthogonal to the first direction (D1), and includes a casing 21, a holding subunit 22, and an actuation subunit 23.

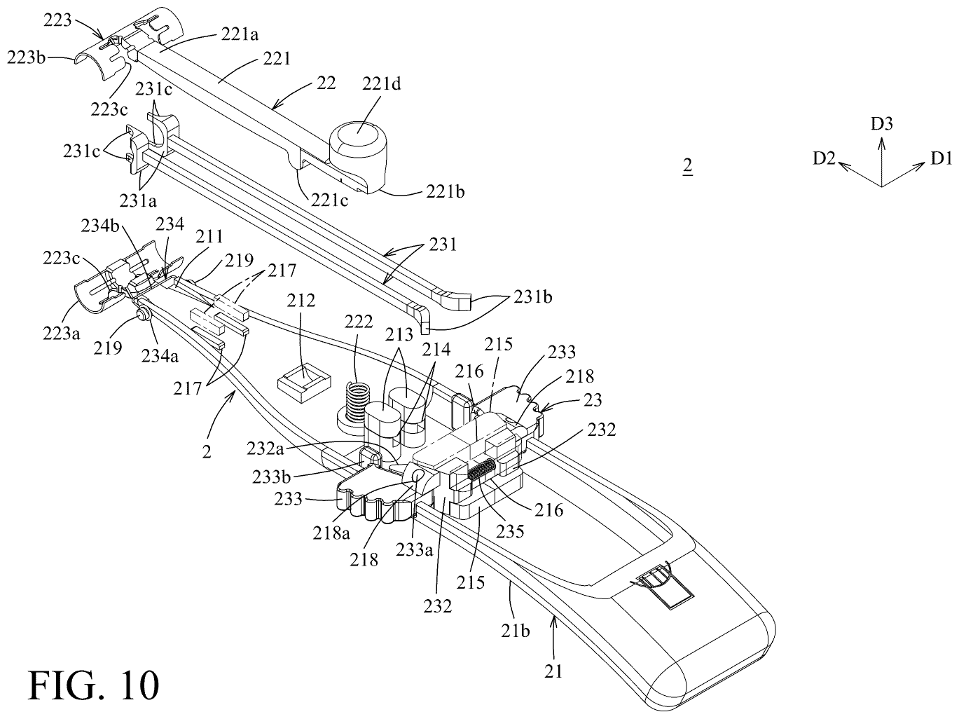

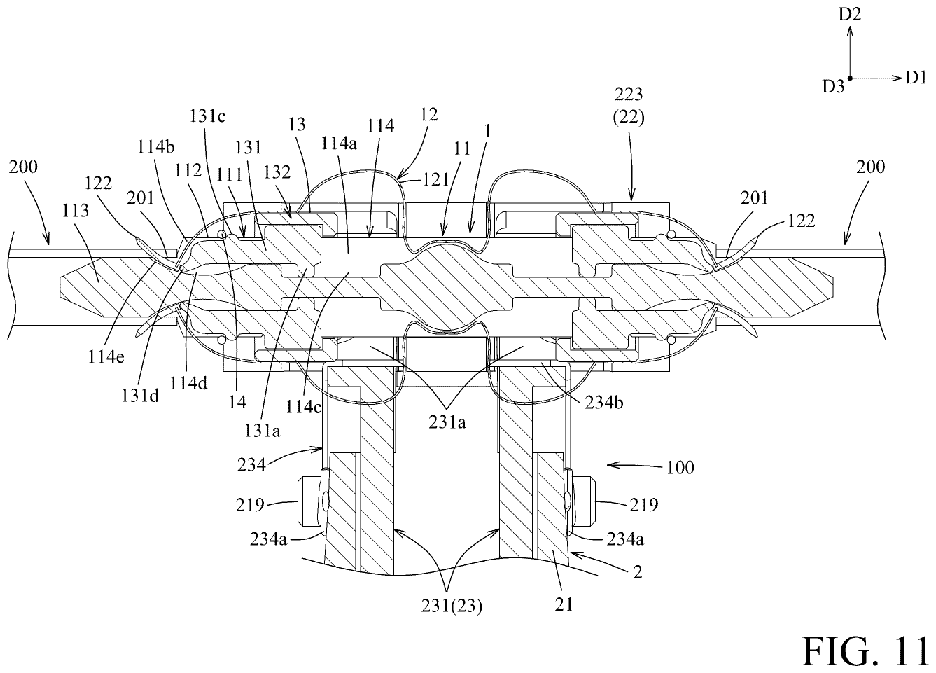

The casing 21 has an upper casing portion 21a, a lower casing portion 21b, a front casing opening 211, a fulcrum portion 212, two first refraining structures 213, two first rails 214, two second refraining structures 215, two second rails 216, a plurality of auxiliary refraining structures 217, two rotation connecting structures 218, and two lower casing protrusions 219. The lower casing portion 21b is located below the upper casing portion 21a in an up-down direction (D3) orthogonal to the first direction (D1) and the front-rear direction (D2). The front casing opening 211 is located at a front end of the casing 21, and is defined by the upper and lower casing portions 21a, 21b. The fulcrum portion 212 is connected to an inner surface of the lower casing portion 21b. The first refraining structures 213 are spaced apart from each other in the first direction (D1), and each of the first refraining structures 213 interconnects the upper and lower casing portions 21a, 21b, and is formed with a respective one of the first rails 214. Each of the first rails 214 extends in the front-rear direction (D2). The second refraining structures 215 are located behind the first refraining structures 213 in the front-rear direction (D2), are respectively connected to an inner surface of the upper casing portion 21a and the inner surface of the lower casing portion 21b, and are respectively formed with the second rails 216. Each of the second rails 216 extends in the first direction (D1). The auxiliary refraining structures 217 are located in front of the first refraining structures 213. A portion of the auxiliary refraining structures 217 is connected to the inner surface of the upper casing portion 21a, and the remaining portion of the auxiliary refraining structures 217 is connected to the inner surface of the lower casing portion 21b. The second refraining structures 215 are located between the rotation connecting structures 218 in the first direction (D1). The rotation connecting structures 218 are connected to the inner surface of the upper casing portion 21a, and each of the rotation connecting structures 218 has a rotation hole 218a. The lower casing protrusions 219 are adjacent to a front end of the lower casing portion 21b, and protrude away from each other in the first direction (D1). It is noted that, in FIG. 10, because the upper casing portion 21a is not shown, the second refraining structure 215 that is connected to the inner surface of the upper casing portion 21a and the second rail 216 thereof, and the portion of the auxiliary refraining structures 217 that is connected to the inner surface of the upper casing portion 21a are depicted in dot-dashed lines, while the rotation connecting structures 218 are depicted in solid lines.

The holding subunit 22 is connected to the casing 21, and has an operation rod 221, a holding resilient member 222, and a holding portion 223. The operation rod 221 is movably disposed on the casing 21. The holding resilient member 222 is located between the operation rod 221 and the casing 21. The holding portion 223 is located at the front end of the casing 21 and is operable to hold the suturing unit 1. The operation rod 221 has a connecting section 221a, an operation section 221b, and a fulcrum section 221c. The connecting section 221a and the operation section 221b are respectively located at a front end and a rear end of the operation rod 221. The fulcrum section 221c is rotatably mounted to the fulcrum portion 212 of the casing 21, is located between the connecting section 221a and the operation 221b in the front-rear direction (D2), and is rotatable about a rotation axis (A) that extends in the first direction (D1). The operation section 221b of the operation rod 221 includes a holding operation button 221d that is exposed from the casing 21. Specifically, the holding operation button 221d upwardly protrudes out of the upper casing portion 21a. The holding resilient member 222 is located between the operation section 221b of the operation rod 221 and the lower casing portion 21b of the casing 21.

The holding portion 223 of the holding subunit 22 includes a first holding body 223a that is connected to the front end of the lower casing portion 21b of the casing 21, a second holding body 223b that is connected to the connecting section 221a of the operation rod 221 and that is located above the first holding body 223a in the up-down direction (D3), and a plurality of through holes 223c. Each of the first holding body 223a and the second holding body 223b of the holding portion 223 is formed with at least one of the through holes 223c. The first and second holding bodies 223a, 223b are operable to cooperatively hold the suturing unit 1.

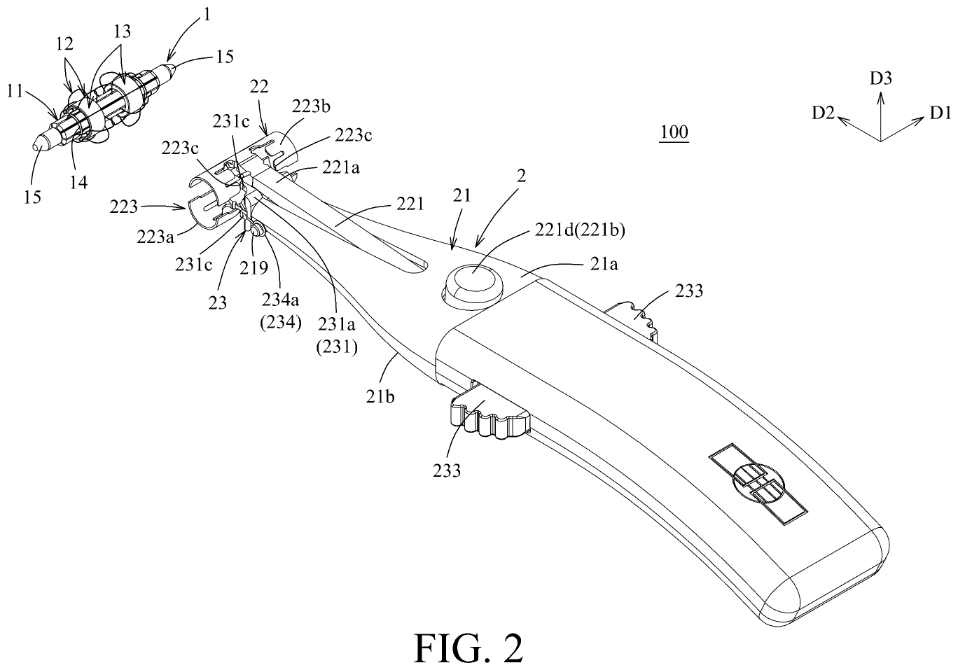

The holding resilient member 222 resiliently biases the operation section 221b upwardly such that the second holding body 223b is urged to move downwardly toward the first holding body 223a and cooperate with the first holding body 223a to hold the suturing unit 1. Specifically, by virtue of the fulcrum section 221c and the fulcrum portion 212, the connecting section 221a is urged to move downwardly when the holding resilient member 222 resiliently biases the operation section 221b upwardly, and thus the second holding body 223b that is connected to the connecting section 221a is urged to move downwardly toward the first holding body 223a. When the holding operation button 221d of the operation section 221b is pressed downwardly, the holding resilient member 222 is compressed, the connecting section 221a is urged to move upwardly by virtue of the fulcrum section 221c and the fulcrum portion 212, and the second holding body 223b that is connected to the connecting section 221a is urged to move upwardly away from the first holding body 223a such that the suturing unit 1 is released from the first holding body 223a and the second holding body 223b (see FIG. 2). It is noted that, in the first embodiment, the operation section 221b and the casing 21 cooperatively hold the holding resilient member 222 therebetween so that the holding resilient member 222 may provide a restoring force to resiliently bias the operation section 221b upwardly and urge the connecting section 221a to move downwardly. However, in one embodiment, the holding resilient member 222 may be connected to the casing 21 and any point of the operation rod 221 as long as the holding resilient member 222 may provide the restoring force to resiliently bias the operation section 221b upwardly and urge the connecting section 221a to move downwardly.

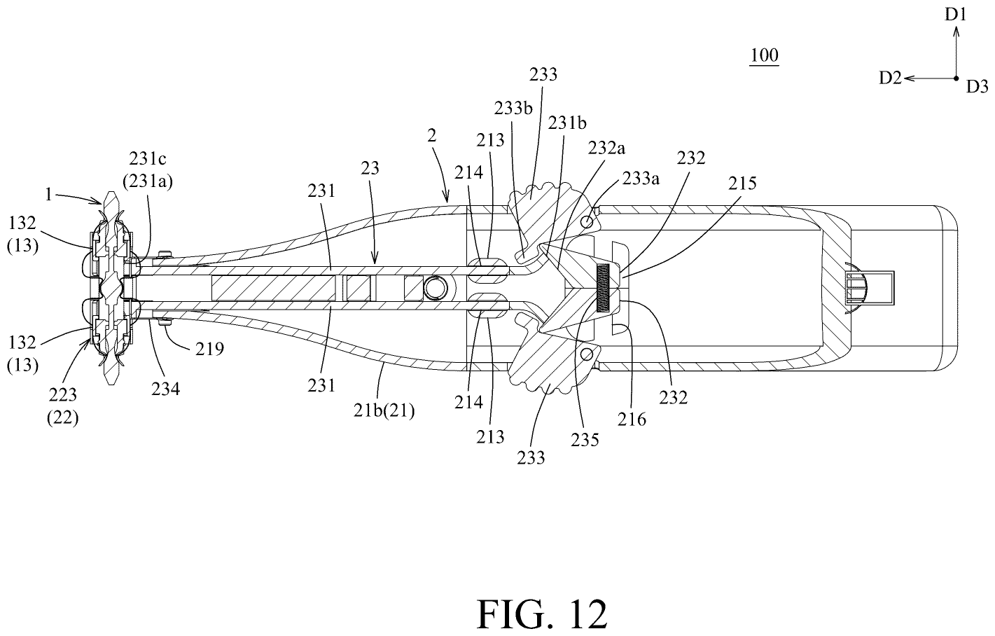

Referring further to FIG. 12, in cooperation with FIGS. 1 and 8 to 10, the actuation subunit 23 is mounted to the casing 21, is substantially located in the casing 21, and is convertible between an initial state (see FIG. 8) and an actuation state (see FIG. 12). The actuation subunit 23 includes two pushing rods 231, two sliding blocks 232, two actuation operation buttons 233, a first resilient member 234, and a second resilient member 235. Each of the pushing rods 231 is elongated in the front-rear direction (D2), and a rear portion thereof is slidably disposed on a respective one of the first rails 214 of the casing 21. The sliding blocks 232 are slidably disposed on the second rails 216, are respectively adjacent to rear ends of the pushing rods 231, and are located between the actuation operation buttons 233 in the first direction (D1). The actuation operation buttons 233 are movably disposed on the casing 21, and a portion of each of the actuation operation buttons 233 is exposed from the casing 21. The first resilient member 234 is disposed on the casing 21, and abuts against front ends of the pushing rods 231. The second resilient member 235 is located between the sliding blocks 232.

The portion of the auxiliary refraining structures 217 that is connected to the upper casing portion 21a and the remaining portion of the auxiliary refraining structures 217 that is connected to the lower casing portion 21b cooperatively refrain front portions of the pushing rods 231 from moving in the up-down direction (D3) so that the pushing rods 231 may slide relative to the first rails 214 more steadily. The pushing rods 231 extend forwardly through the front casing opening 211 of the casing 21 so that the pushing rods 231 are respectively adjacent to the pushing rings 132 of the pushing subunits 13. Each of the pushing rods 231 has a pushing section 231a that is located at the front end thereof, that is located outside of the casing 21, and that is operable to move toward the holding portion 223 of the holding subunit 22 to push the pushing ring 132 of the respective one of the pushing subunits 13, and a first rod surface 231b that is located at the rear end thereof. The first rod surfaces 231b of the pushing rods 231 face away from each other, and a distance therebetween increases forwardly. The pushing section 231a of each of the pushing rods 231 is substantially U-shaped and opens forwardly. The pushing section 231a of each of the pushing rods 231 has two pushing surfaces 231c (see FIG. 10) each of which is oblique to the front-rear direction (D2). Each of the pushing surfaces 231c of one of the pushing rods 231 faces away from a respective one of the pushing surfaces 231c of the other one of the pushing rods 231 in the first direction (D1), and a distance between each of the pushing surfaces 231c of one of the pushing rods 231 and the respective one of the pushing surfaces 231c of the other one of the pushing rods 231 decreases forwardly. In the first embodiment, each of the pushing surfaces 231c is configured to be a concave surface. However, in one embodiment, each of the pushing surfaces 231c may be, but not limited to, a convex surface, or a flat surface that is oblique to the front-rear direction (D2). Each of the sliding blocks 232 has a first block surface 232a that faces the first rod surface 231b of the respective one of the pushing rods 231. The first block surfaces 232a of the sliding blocks 232 face each other, and a distance therebetween increases forwardly.

The actuation operation buttons 233 are respectively mounted to the rotation connecting structures 218 of the casing 21. Each of the actuation operation buttons 233 includes a rotation rod 233a that is located at a rear portion thereof, that extends upwardly into the rotation hole 218a of the respective one of the rotation connecting structures 218, and that is rotatable relative to the rotation hole 218a of the rotation connecting structure 218, and an abutting block 233b that extends forwardly. The casing 21 has two inner lateral surfaces that are spaced apart from each other in the first direction (D1) and that face each other. The abutting blocks 233b of the actuation operation buttons 233 respectively abut against the inner lateral surfaces of the casing 21.

The first resilient member 234 has two casing-connecting ring sections 234a that are located at two opposite ends thereof, and that are respectively disposed on the lower casing protrusions 219 of the casing 21, and a resilient bar section 234b that interconnects the casing-connecting ring sections 234a, and that abuts against front sides of the pushing sections 231a of the pushing rods 231.

Operation of the suturing device 100 is described below.

FIGS. 1, 5 and 8 illustrate the suturing unit 1 already being held by the holding subunit 22 of the actuation unit 2. Then, the groove covers 15 are removed from the insertion sections 113 of the carrying portions 111 of the suturing aid rod 11, and the insertion sections 113 are respectively inserted into the connecting openings 201 of the tubular objects 200. At this time, the pushing subunits 13 are in the initial position. The pushing subunits 13 are proximate to the center of the suturing aid rod 11. The annular resilient members 14 block the blocking protrusion sections 131c of the sliding plates 131 so that the pushing subunits 13 are refrained from moving away from each other at this time. The actuation subunit 23 is free from external forces and is in the initial state. When the actuation subunit 23 in the initial state, the pushing rods 231 are urged by the first resilient member 234 to be in a first rod position, and the sliding blocks 232 are urged by the second resilient member 235 to be in a first block position.

Referring to FIGS. 11 and 12 again, afterwards, when the actuation operation buttons 233 are pushed toward each other in the first direction (D1) by the external forces, the sliding blocks 232 are respectively pushed by the actuation operation buttons 233, slide toward each other in the first direction (D1), cooperatively compress the second resilient member 235, and respectively push the first rod surfaces 231b of the pushing rods 231 via the first block surfaces 232a thereof such that the pushing rods 231 are moved forwardly and that the actuation subunit 23 are converted from the initial state to the actuation state. At this time, the sliding blocks 232 are urged by the actuation operation buttons 233 to be in a second block position.

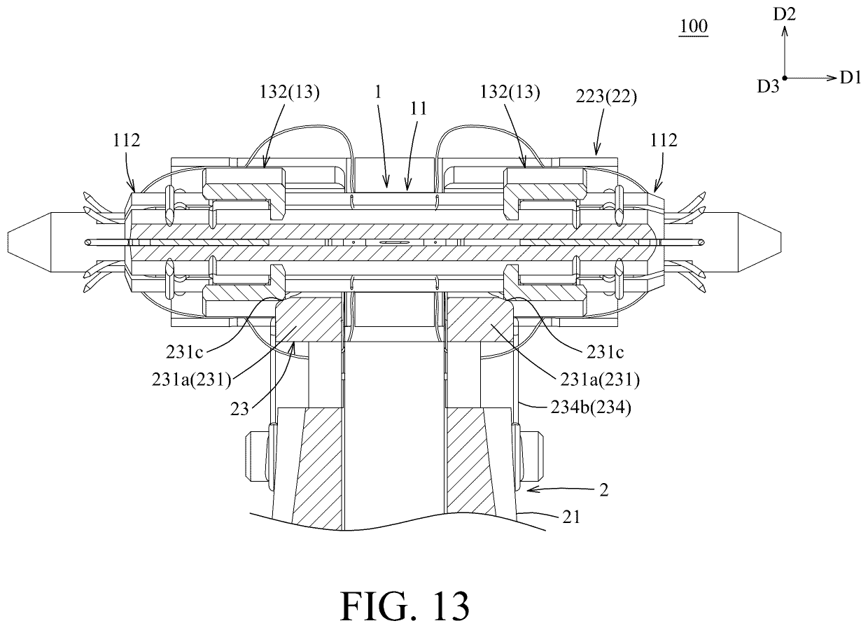

Referring further to FIG. 13, during conversion of the actuation subunit 23 from the initial state to the actuation state, the pushing rods 231 are moved forwardly such that the pushing sections 231a of the pushing rods 231 pass through the through holes 223c of the holding portion 223, and the pushing surfaces 231c of the pushing rods 231 push the pushing subunits 13 away from each other such that the pushing subunits 13 are moved from the initial position to the puncture position. Each of the annular resilient members 14 abuts against the blocking protrusion sections 131c of the sliding plates 131 of a respective one of the pushing subunits 13 so that when the pushing subunits 13 are moved from the initial position to the puncture position, a predetermined force has to be exerted on the pushing ring 132 of each of the pushing subunits 13 to urge the pushing ring 132 of each of the pushing subunits 13 to push the sliding plates 131 of the pushing subunit 13 and the respective one of the annular resilient members 14 against a retarding force generated by the respective one of the annular resilient members 14. That is to say, the external forces that are exerted on the actuation operation buttons 233 have to be large enough so that the sliding plates 131 and the annular resilient members 14 may be pushed together. When the pushing subunits 13 are moved from the initial position to the puncture position, the pushing ring 132 of each of the pushing subunits 13 pushes the sliding plates 131 of the pushing subunit 13 and the respective one of the annular resilient members 14 away from the center of the suturing aid rod 11 such that the sliding plates 131 of the pushing subunit 13 slide toward the first accommodating parts 114b of the grooves 114 of the respective one of the carrying portions 111, and that the needle-pushing sections 131d of the sliding plates 131 of the pushing subunits 13 respectively push the suture needles 122 of the sewing subunits 12 and urge the suture needles 122 to respectively slide along the curved areas 114d of the grooves 114, to respectively protrude from the needle openings 114e of the grooves 114, and to puncture the tubular objects 200. At this time, the pushing rods 231 are urged by the sliding blocks 232 to be in a second rod position.

Referring to FIGS. 8 and 11 to 13 again, when the actuation subunit 23 is in the actuation state, the second resilient member 235 is resiliently compressed by the sliding blocks 232, and the pushing sections 231a of the pushing rods 231 push the resilient bar section 234b of the first resilient member 234 forwardly such that the resilient bar section 234b of the first resilient member 234 is resiliently stretched. After the tubular objects 200 are punctured by the suture needles 122, the external forces may be stopped being exerted on the actuation operation buttons 233. When the actuation operation buttons 233 are free from the external forces, the first resilient member 234 exerts a first restoring force on each of the pushing rods 231 such that the pushing rods 231 slide rearwardly, and the second resilient member 235 exerts a second restoring force on each of the sliding blocks 232 such that the sliding blocks 232 slide away from each other and respectively urge the actuation operation buttons 233 to move away from each other, thereby converting the actuation subunit 23 from the actuation state to the initial state. At this time, the pushing rods 231 return to the first rod position, and the sliding blocks 232 return to the first block position.

Referring to FIG. 2 again, afterwards, when the holding operation button 221d is depressed (i.e., the operation section 221b is pressed downwardly), the suturing unit 1 is released from the holding portion 223 of the holding subunit 22. Then, the suturing aid rod 11, the pushing subunits 13, and the annular resilient members 14 may be removed from the sewing subunits 12 and the tubular objects 200. After the suturing aid rod 11, the pushing subunits 13, and the annular resilient members 14 are removed, each of the suture threads 121 that extends through the tubular objects 200 is pulled to urge the tubular objects 200 to move toward each other and is tied in a knot, thereby completing a process of sewing the tubular objects 200 together.

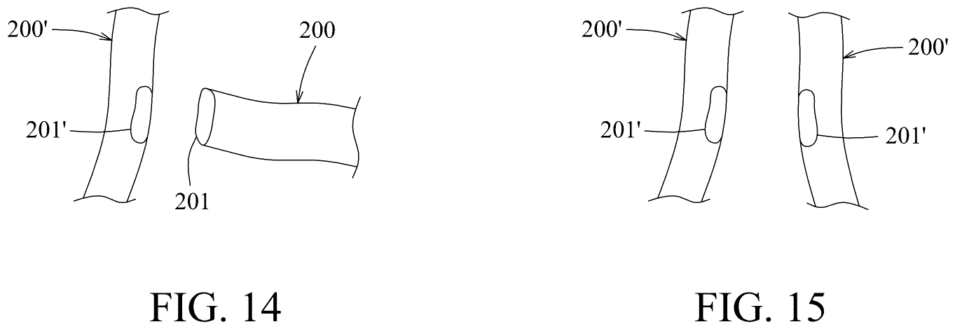

It is noted that, the suturing device 100 may be adapted for sewing different tubular objects. For example, referring to FIG. 14, the suturing device may be adapted for sewing the tubular object 200 that has a longitudinal opening 201, and another tubular object 200′ that has a lateral opening 201′. In addition, referring to FIG. 15, the suturing device may be adapted for sewing two tubular objects 200′ each of which has a lateral opening 201′.

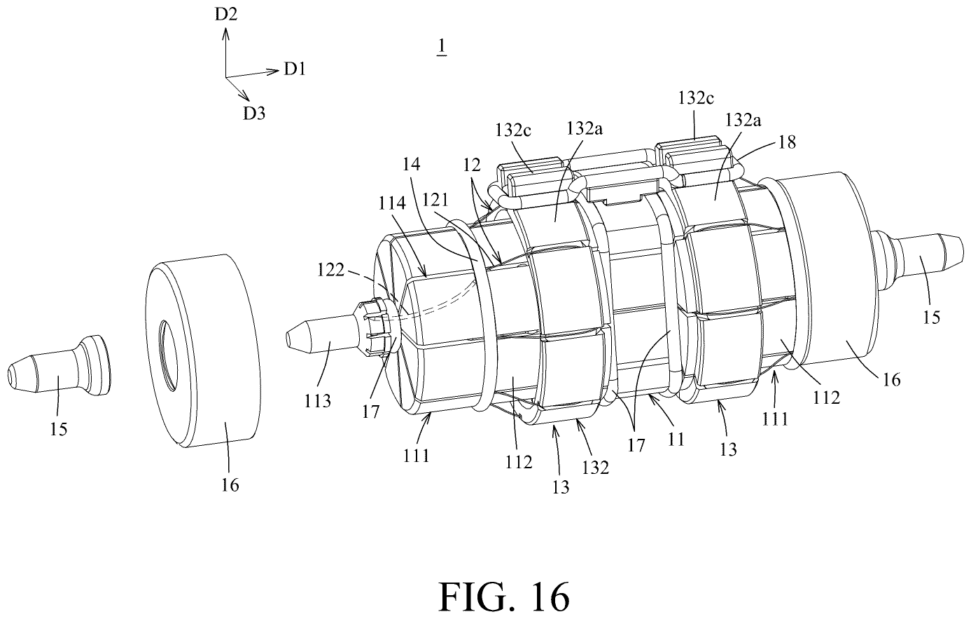

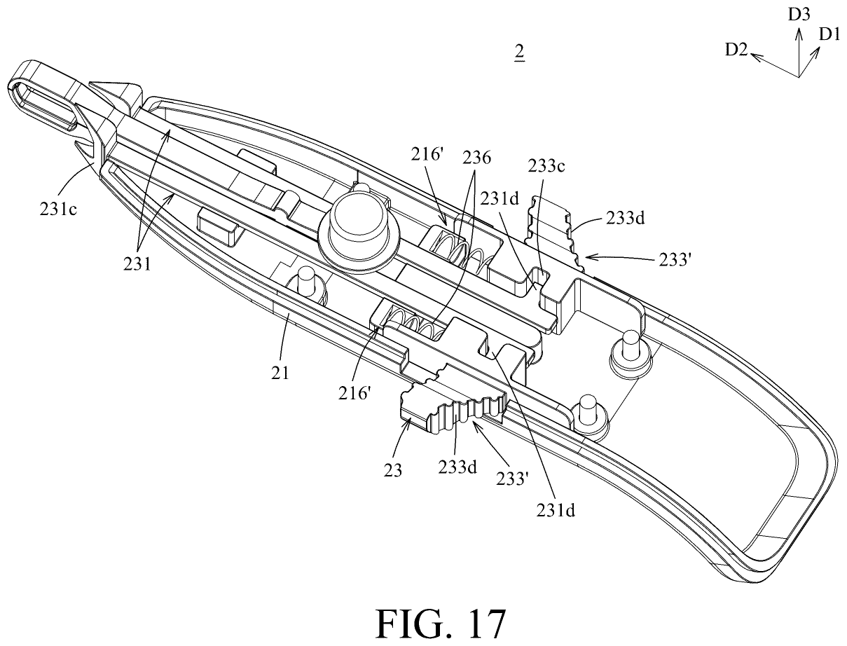

Referring to FIGS. 16 and 17, a second embodiment of the suturing device 100 according to the disclosure includes differences lying in the suturing unit 1 and the actuation unit 2. In the second embodiment, the pushing ring 132 of each of the pushing subunits 13 has a positioning protrusion section 132c that protrudes from the surrounding section 132a of the pushing subunit 13. The suturing unit 1 further includes two protective covers 16, a plurality of suture surrounding rings 17, and a ring-positioning member 18. Each of the protective covers 16 surrounds an end of the carrying section 112 of a respective one of the carrying portions 111 that is adjacent to the insertion section 113 of the carrying portion 111 so that the suture needles 122 of the sewing subunits 12 are prevented from leaving the grooves 114 of the carrying portions 111. Each of the suture surrounding rings 17 surrounds the suturing aid rod 11 and the suture threads 121 of the sewing subunits 12 so that the suture threads 121 and the suturing aid rod 11 are prevented from separation. The ring-positioning member 18 surrounds the positioning protrusion sections 132c of the pushing rings 132 so that the pushing subunits 13 are kept in the initial position.

Moreover, each of the pushing rods 231 further has a coupling protrusion section 231d that is located at the rear end thereof. The coupling protrusion sections 231d of the pushing rods 231 protrude away from each other in the first direction (D1). The casing 21 further includes two operation rails 216′ each of which extends in the front-rear direction (D2). The actuation subunit 23 further includes two actuation operation buttons 233′ that are respectively and slidably disposed on the operation rails 216′ of the casing 21, that are respectively adjacent to the rear ends of the pushing rods 231, and that are exposed from the casing 21. Specifically, a portion of each of the actuation operation buttons 233′ is exposed from the casing 21. In addition, the actuation subunit 23 further includes two button resilient members 236 each of which is located between the casing 21 and a respective one of the actuation operation buttons 233′. Each of the actuation operation buttons 233′ has a coupling groove section 233c that is coupled to the coupling protrusion section 231d of the respective one of the pushing rods 231, and an operation surface 233d that is exposed from the casing 21. The operation surfaces 233d of the actuation operation buttons 233′ face away from each other in the first direction (D1) and a distance thereof increases forwardly so that when the operation surfaces 233d are pushed toward each other in the first direction (D1) by external forces, the actuation operation buttons 233′ are urged to slide forwardly. Because the coupling groove sections 233c are respectively coupled to the coupling protrusion sections 231d of the pushing rods 231, when the actuation operation buttons 233′ are pushed to slide forwardly by the external forces, the pushing rods 231 are urged to move forwardly such that the actuation subunit 23 are converted from the initial state to the actuation state. Furthermore, when the actuation operation buttons 233′ are pushed to slide forwardly by the external forces, the button resilient members 236 are respectively and resiliently compressed by the actuation operation buttons 233′. When the actuation operation buttons 233′ are free from the external forces, each of the button resilient members 236 exerts a button restoring force to the respective one of the actuation operation buttons 233′ to urge the respective one of the actuation operation buttons 233′ to slide rearwardly such that the pushing rods 231 are urged to move rearwardly and the actuation subunit 23 are converted from the actuation state to the initial state.

In addition, in the second embodiment, each of the pushing rods 231 has only one pushing surface 231c, and the pushing surface 231c of each of the pushing rods 231 is configured to be a flat surface that is oblique to the front-rear direction (D2).

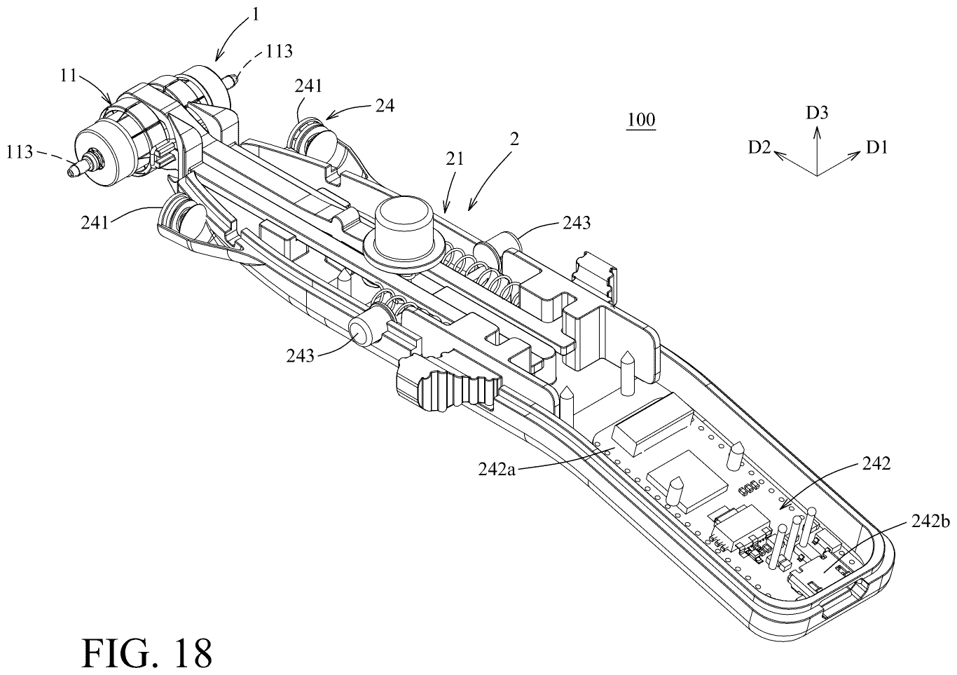

Referring to FIG. 18, a third embodiment of the suturing device 100 according to the disclosure includes differences lying in the actuation unit 2. In the third embodiment, the actuation unit 2 further includes a video subunit 24. The video subunit 24 includes two shooting cameras 241, an image processing circuit module 242, and two camera switches 243. Each of the shooting cameras 241 is disposed on the casing 21 and faces forwardly so that each of the shooting cameras 241 is operable to record movements of the suturing unit 1. Specifically, the shooting cameras 241 respectively face the insertion sections 113 of the carrying portions 111 of the suturing aid rod 11 so that the shooting cameras 241 are operable to record the movements of the suturing unit 1. The image processing circuit module 242 is disposed in the casing 21 and is electrically coupled to the shooting cameras 241. The camera switches 243 are disposed on the casing 21, are electrically coupled to the image processing circuit module 242, and are operable to respectively control the shooting cameras 241 (i.e., each of the camera switches 243 is operable to turn on and off the respective one of the shooting cameras 241). A portion of each of the camera switches 243 is exposed from the casing 21. The image processing circuit module 242 includes an image processing circuit board 242a, and a connecting port 242b that is disposed on the image processing circuit board 242a. A portion of the connecting port 242b is exposed from the casing 21. The image processing circuit board 242a may include, for example, a rechargeable battery (not shown), a Wi-Fi submodule (not shown) that is capable of transmitting images, and a computer memory (not shown), and is installed with an image processing firmware. The connecting port 242b is adapted to be connected to a charger (not shown) so that the rechargeable battery of the image processing circuit board 242a may be recharged.

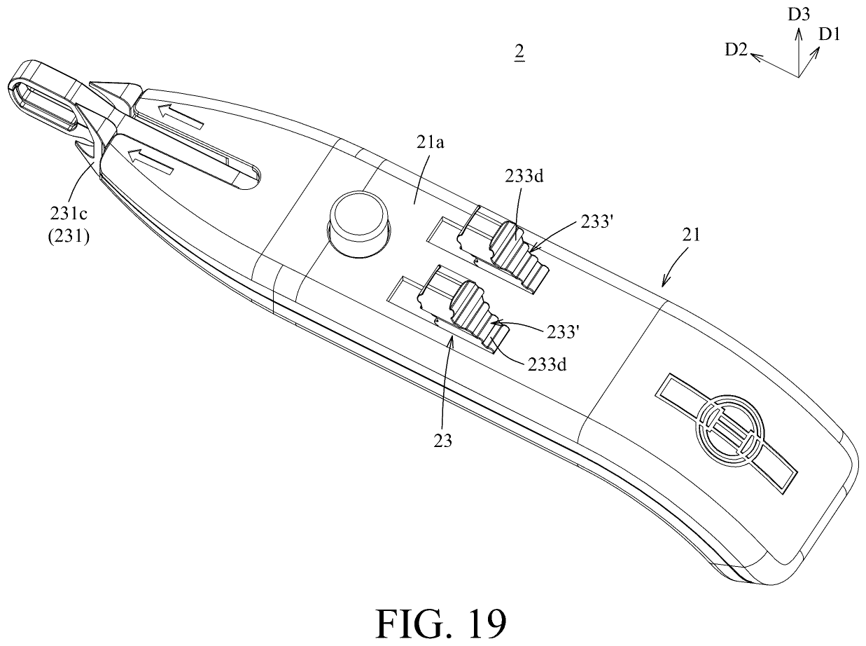

Referring to FIG. 19, a fourth embodiment of the suturing device 100 according to the disclosure includes differences lying in the actuation operation buttons 233′ of the actuation unit 2. In the fourth embodiment, a portion of each of the actuation operation buttons 233′ is exposed upwardly from the casing 21, and is elongated in the third direction (D3). It is noted that, in a modification of the fourth embodiment, structure of each of the pushing rods 231 other than the actuation operation buttons 233′ may be the same as that of the second embodiment according to actual requirements. In another modification of the fourth embodiment, according to actual requirements, structure of each of the pushing rods 231 other than the actuation operation buttons 233′ may differ from that of the second embodiment.

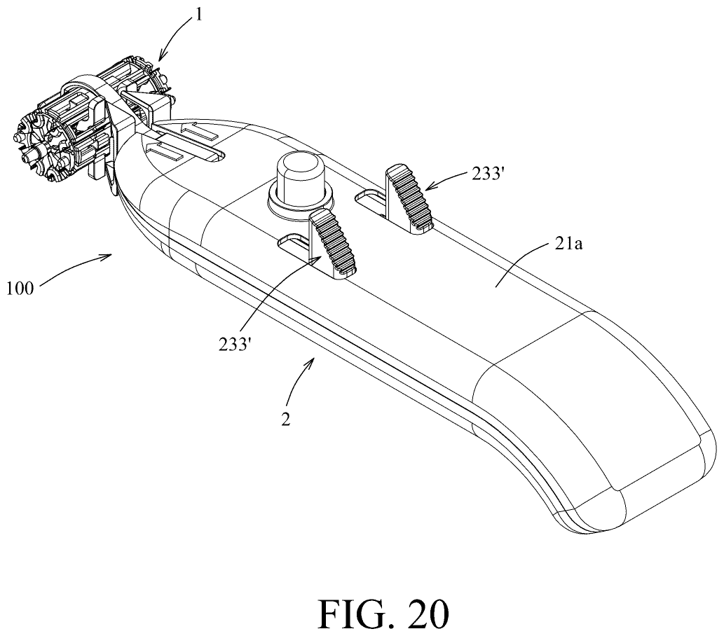

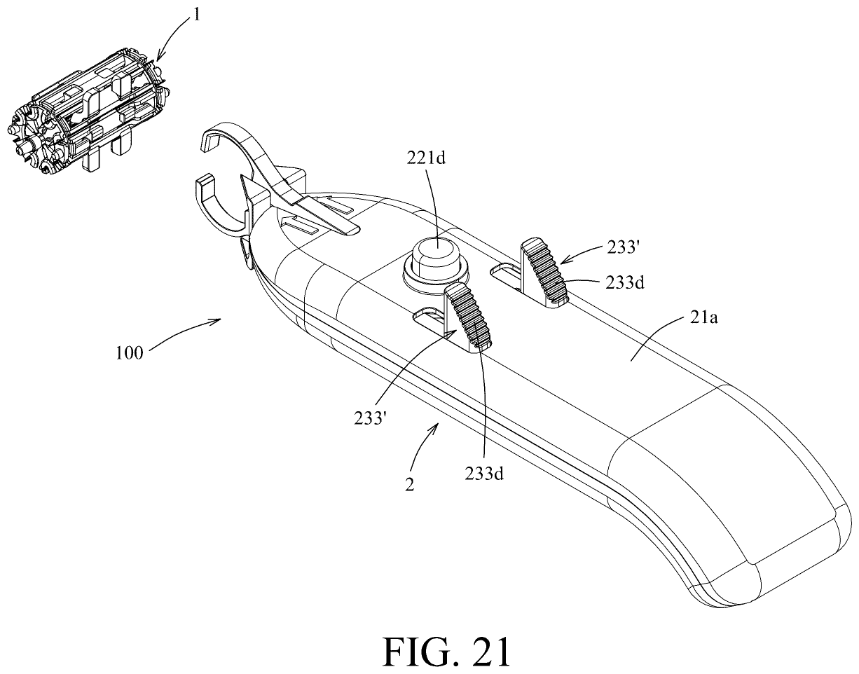

Referring to FIGS. 20 and 21, a fifth embodiment of the suturing device 100 according to the disclosure includes differences lying in the suturing unit 1. The actuation operation buttons 233′ of the fifth embodiment are similar to the actuation operation buttons 233′ of the fourth embodiment. The operation surfaces 233d of the actuation operation buttons 233′ of each of the fourth and fifth embodiments face rearwardly. In each of the fourth and fifth embodiments, the holding operation button 221d upwardly protrudes out of the upper casing portion 21a, and the portion of each of the actuation operation buttons 233′ is exposed upwardly from the casing 2 so that it may be easier for a user to understand how to operate the suturing device 100.

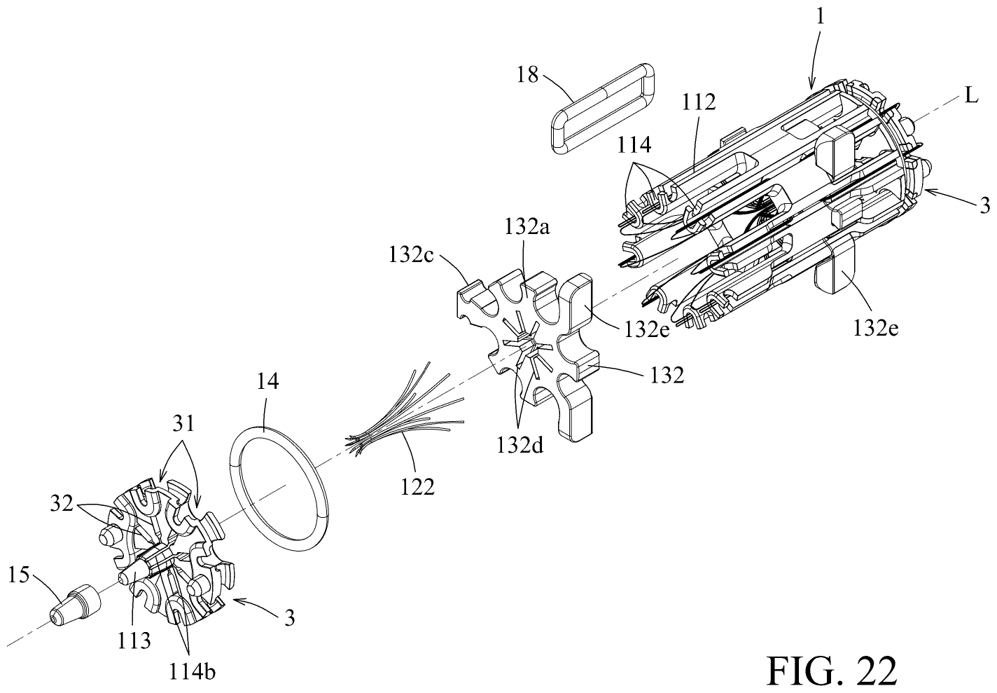

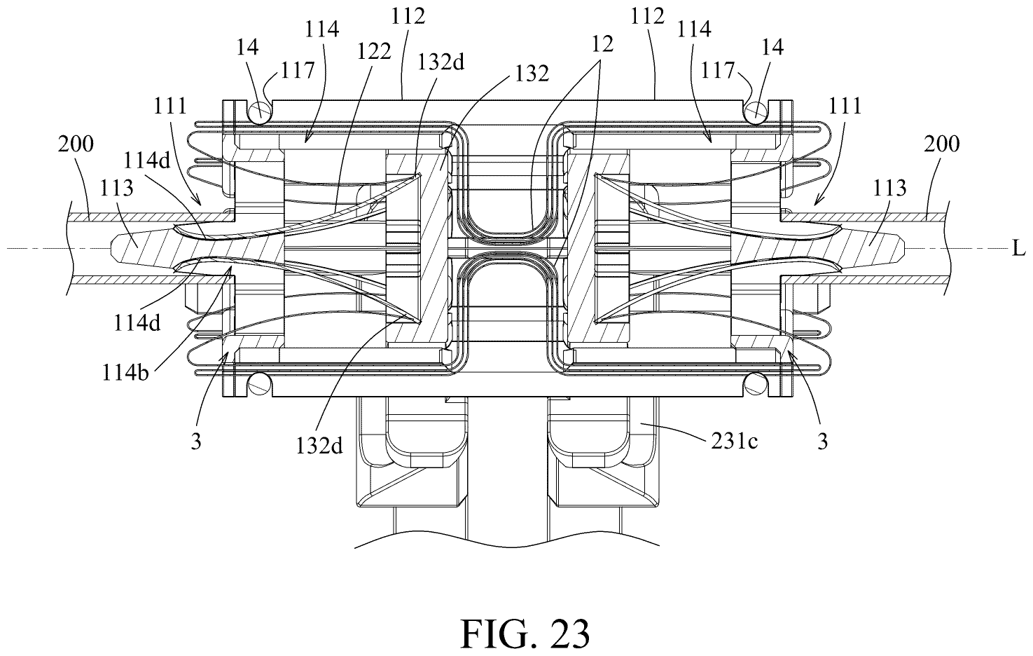

Referring further to FIGS. 22 to 24, in the fifth embodiment, the suturing unit 1 further includes two positioning circular bodies 3 each of which is disposed between the carrying section 112 and the insertion section 113 of a respective one of the carrying portions 111. A periphery of each of the positioning circular bodies 3 is formed with a plurality of recesses 31. A quantity of the recesses 31 of each of the positioning circular bodies 3 is the same as a quantity of the grooves 114 of the respective one of the carrying portions 111. Each of the positioning circular bodies 3 has one of male and female engaging structures, and each of the carrying portions 111 has another one of the male and female engaging structures so that the positioning circular bodies 3 may be respectively coupled to the carrying portions 111. Each of the positioning circular bodies 3 has a plurality of through openings 32 that are disposed about the axis (L) and that correspond in position to the first accommodating parts 114b of the grooves 114 of the respective one of the carrying portions 111. The pushing ring 132 of each of the pushing units 13 further has a plurality of needle-abutting grooves 132d that are disposed about the axis (L) and that correspond in position to the first accommodating parts 114b of the grooves 114 of the respective one of the carrying portions 111, and a quantity of the needle-abutting grooves 132d of each of the pushing units 13 is the same as the quantity of the grooves 114 of the respective one of the carrying portions 111. One of the suture needles 122 of each of the sewing subunits 12 abuts against a respective one of the needle-abutting grooves 132d of one of the pushing units 13, and another one of the suture needles 122 of each of the sewing subunits 12 abuts against a respective one of the needle-abutting grooves 132d of another one of the pushing units 13. For each of the carrying portions 111, the curved area 114d of the first accommodating part 114b of each of the grooves 114 is located at the insertion section 113. The one of the suture needles 122 of each of the sewing subunits 12 is disposed on the curved area 114d of the first accommodating part 114b of a respective one of the grooves 114 of one of the carrying portions 111, and has a cross-section that is shaped to correspond to a cross-section of the curved area 114d of the first accommodating part 114b of the respective one of the grooves 114 of the one of the carrying portions 111. The another one of the suture needles 122 of each of the sewing subunits 12 is disposed on the curved area 114d of the first accommodating part 114b of a respective one of the grooves 114 of another one of the carrying portions 111, and has a cross-section that is shaped to correspond to a cross-section of the curved area 114d of the first accommodating part 114b of the respective one of the grooves 114 of the another one of the carrying portions 111. Therefore, when the pushing subunits 13 are in the initial position, a pointed end of the one of the suture needles 122 of each of the sewing subunits 12 is located in the first accommodating part 114b of the respective one of the grooves 114 of the one of the carrying portions 111, and a pointed end of the another one of the suture needles 122 of each of the sewing subunits 12 is located in the first accommodating part 114b of the respective one of the grooves 114 of the another one of the carrying portions 111 (see FIG. 23).

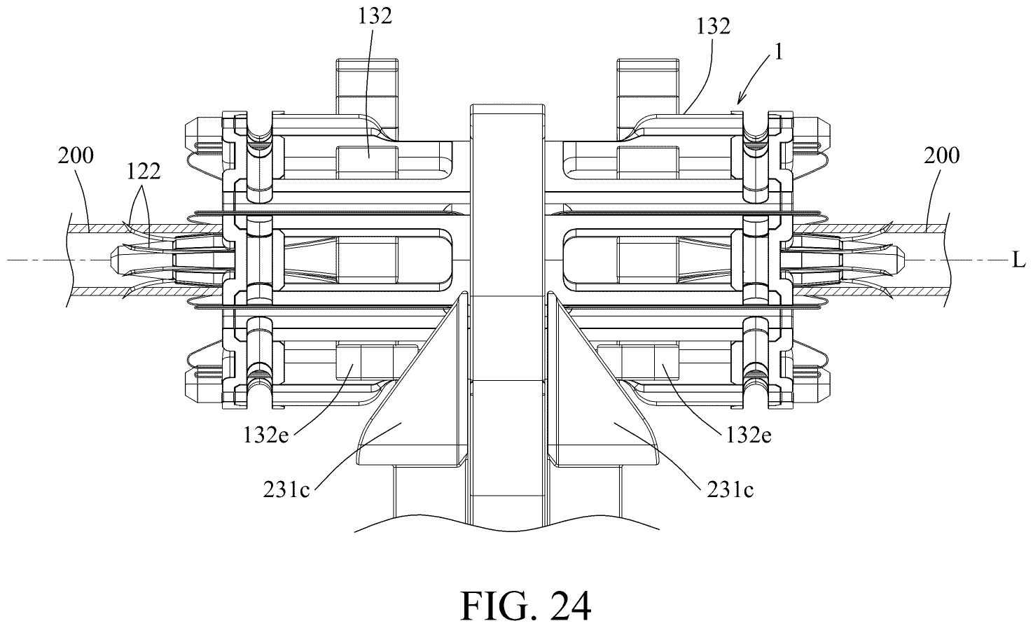

The pushing ring 132 of each of the pushing subunits 13 further has a pushing protrusion section 132e that protrudes from one side of the surrounding section 132a of the pushing subunit 13 opposite to the positioning protrusion section 132c of the pushing subunit 13. During conversion of the actuation subunit 23 from the initial state to the actuation state, the pushing surfaces 231c of the pushing rods 231 push the pushing protrusion sections 132e of the pushing rings 132 of the pushing subunits 13 away from each other such that the pushing subunits 13 are moved from the initial position to the puncture position. At this time, the needle-abutting grooves 132d of each of the pushing units 13 respectively push the suture needles 122 of the sewing subunits 12 and urge the suture needles 122 to respectively protrude from the needle openings 114e of the grooves 114, and to puncture the tubular objects 200 (see FIG. 24).

It is noted that, in order to prevent the suture needles 122 and the suture threads 121 of the sewing subunits 12 from leaving the grooves 114 of the carrying portions 111 when the pushing subunits 13 are in the initial position, the suturing unit 1 of the fifth embodiment also includes the groove covers 15 that respectively surround the insertion sections 113 of the suturing aid rod 11, and the ring-positioning member 18 that surrounds the positioning protrusion sections 132c of the pushing rings 132. Moreover, the suturing unit 1 of the fifth embodiment further has two positioning portions 117 each of which is defined by the grooves 114 of a respective one of the carrying portions 111, is adjacent to a respective one of the positioning circular bodies 3, and is in communication with the grooves 114 of the respective one of the carrying portions 111. Specifically, each of the positioning portions 117 cooperates with the respective one of the positioning circular bodies 3 to define a circular groove that surrounds the positioning circular body 3. The annular resilient members 14 are respectively disposed on the positioning portions 117 (i.e., respectively disposed in the circular grooves). An inner circumferential edge of each of the annular resilient members 14 is located within the grooves 114 of a respective one of the carrying portions 111. Each of the annular resilient members 14 extends through the grooves 114 of the respective one of the carrying portions 111, and surrounds the sewing subunits 12 so as to prevent the suture threads 121 from leaving the grooves 114 of the carrying portions 111 when the pushing subunits 13 are in the initial position.

In summary, by virtue of the pushing ring 132 of each of the pushing subunits 13 pushing the sliding plates 131 of the pushing subunit 13, and by virtue of the sliding plates 131 of the pushing subunits 13 respectively pushing the suture needles 122 of the sewing subunits 12 and urging the suture needles 122 to respectively slide along the curved areas 114d of the grooves 114, the suture needles 122 of the sewing subunits 12 may respectively protrude from the needle openings 114e of the grooves 114 and puncture the tubular objects 200. Afterwards, the tubular objects 200 may be simply sewn together by pulling each of the suture threads 121 and tying each of the suture threads 121 in a knot. Therefore, via the suturing device 100, a surgical anastomosis for connecting two tubular body structures that rupture may be relatively easy to perform, and may be timesaving.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A suturing device adapted for sewing two tubular objects together, each of the tubular objects having a connecting opening, the suturing device comprising:
a suturing unit including
a suturing aid rod that is elongated in a first direction and that has two carrying portions opposite to each other in the first direction, each of the carrying portions having a carrying section that is proximate to another one of the carrying portions, an insertion section that is distal from the another one of the carrying portions and that is adapted to be inserted into the connecting opening of a respective one of the tubular object, and a plurality of grooves that are disposed about an axis extending in the first direction, for each of the carrying portions, each of the grooves extending in the first direction and having a first accommodating part that is located at the insertion section, and that has a curved area and a needle opening spatially communicating with the curved area and located at the insertion section, the curved area of the first accommodating part of each of the grooves having two side segments that are spaced apart from each other in the first direction and that are distal from the axis, and a middle segment that interconnects the side segments and that is proximate to the axis,
a plurality of sewing subunits each of which includes a suture thread and two suture needles that are respectively connected to two opposite ends of the suture thread, one of the suture needles of each of the sewing subunits being disposed on the curved area of the first accommodating part of a respective one of the grooves of one of the carrying portions and having a cross-section that is shaped to correspond to a cross-section of the curved area of the first accommodating part of the respective one of the grooves of the one of the carrying portions, the other one of the suture needles of each of the sewing subunits being disposed on the curved area of the first accommodating part of a respective one of the grooves of the other one of the carrying portions and having a cross-section that is shaped to correspond to a cross-section of the curved area of the first accommodating part of the respective one of the grooves of the other one of the carrying portions, and
two pushing subunits each of which is mounted to a respective one of the carrying portions and is slidable relative to the respective one of the carrying portions between an initial position and a puncture position in the first direction, the pushing subunits being proximate to a center of the suturing aid rod when the pushing subunits are in the initial position, the pushing subunits being operable to move away from each other toward the puncture position so that the pushing subunits respectively push the suture needles of the sewing subunits and urge the suture needles to respectively slide along the curved areas of the grooves and respectively protrude out of the needle opening of the grooves, when the pushing subunits are in the puncture position, one of the suture needles of each of the sewing subunits being adapted to puncture one of the tubular objects, and the other one of the suture needles of each of the sewing subunits being adapted to puncture the other one of the tubular objects.

2. The suturing device as claimed in claim 1, wherein:
for each of the carrying portions, each of the grooves further has a second accommodating part that is located at the carrying section;
for each of the carrying portions, the first accommodating part of each of the grooves extends from the insertion section to the carrying section;
each of the pushing subunits of the suturing unit includes a plurality of sliding plates that are respectively disposed in the second accommodating parts of the grooves of the respective one of the carrying portions, and a pushing ring that is slidably mounted to the respective one of the carrying portions and that is operable to push the sliding plates; and
the pushing rings of the pushing subunits are operable to move away from each other toward the puncture position so that the pushing ring of each of the pushing subunits pushes the sliding plates of the pushing subunit toward the first accommodating parts of the grooves of the respective one of the carrying portions, and that the sliding plates of the pushing subunits respectively push the suture needles of the sewing subunits and urge the suture needles to respectively slide along the curved areas of the grooves and respectively protrude out of the needle opening of the grooves.

3. The suturing device as claimed in claim 2, wherein each of the sliding plates has a ring-surrounded section that protrudes out of the second accommodating part of the respective one of the grooves, the pushing ring of each of the pushing subunits having a surrounding section that surrounds the ring-surrounded sections of the sliding plates of the pushing subunit, and a plate-pushing section that is connected to the surrounding section and that is operable to push the ring-surrounded sections of the sliding plates of the pushing subunit.

4. The suturing device as claimed in claim 2, wherein the second accommodating part of each of the grooves has an indented area, each of the sliding plates having a sliding section that extends into the indented area of the respective one of the grooves and that is slidable relative to the indented area of the respective one of the grooves in the first direction.

5. The suturing device as claimed in claim 2, wherein each of the carrying portions of the suturing aid rod further has an annular indentation section that is located at the carrying section and that intersects the second accommodating parts of the grooves of the carrying portion, the suturing unit further including two annular resilient members that are respectively disposed on the annular indentation sections of the carrying portions, each of the sliding plates having a blocking protrusion section that is located between the annular resilient members in the first direction, each of the annular resilient members abutting against the blocking protrusion sections of the sliding plates of a respective one of the pushing subunits so that when the pushing subunits are moved from the initial position to the puncture position, a predetermined force has to be exerted on the pushing ring of each of the pushing subunits to urge the pushing ring of each of the pushing subunits to push the sliding plates of the pushing subunit and the respective one of the annular resilient members.

6. The suturing device as claimed in claim 2, wherein the suturing unit further includes two groove covers that respectively surround the insertion sections of the suturing aid rod, each of the groove covers covering the needle openings of the grooves of a respective one of the carrying portions.

7. The suturing device as claimed in claim 2, wherein the suturing unit further includes two protective covers each of which surrounds an end of the carrying section of a respective one of the carrying portions adjacent to the insertion section of the carrying portion so that the suture needles of the sewing subunits are prevented from leaving the grooves of the carrying portions.

8. The suturing device as claimed in claim 2, wherein the suturing unit further includes a plurality of suture surrounding rings each of which surrounds the suturing aid rod and the suture threads of the sewing subunits so that the suture threads and the suturing aid rod are prevented from separation.

9. The suturing device as claimed in claim 2, wherein the pushing ring of each of the pushing subunits has a positioning protrusion section, the suturing unit further including a ring-positioning member that surrounds the positioning protrusion sections of the pushing rings so that the pushing subunits are kept in the initial position.

10. The suturing device as claimed in claim 1, further comprising:
an actuation unit including
a casing that includes two first rails each of which extends in a front-rear direction orthogonal to the first direction,
a holding subunit that is connected to the casing and that has a holding portion located at a front end of the casing and operable to hold the suturing unit, and
an actuation subunit that is mounted to the casing, that is convertible between an initial state and an actuation state, and that includes two pushing rods each of which is elongated in the front-rear direction, is slidably disposed on a respective one of the first rails of the casing, and has a pushing section located at a front end thereof and operable to push the pushing ring of a respective one of the pushing subunits, the pushing section of each of the pushing rods having a pushing surface that is oblique to the front-rear direction, the pushing surfaces of the pushing rods facing away from each other in the first direction and a distance therebetween decreasing forwardly, during conversion of the actuation subunit from the initial state to the actuation state, the pushing rods being moved forwardly, and the pushing surfaces of the pushing rods respectively pushing the pushing subunits away from each other such that the pushing subunits are moved from the initial position to the puncture position.

11. The suturing device as claimed in claim 10, wherein each of the pushing rods further has a first rod surface that is located at a rear end thereof, the first rod surfaces of the pushing rods facing away from each other and a distance therebetween increasing forwardly, the casing further including a second rail that extends in the first direction, the actuation subunit further including two actuation operation buttons that are movably disposed on the casing and that are exposed from the casing, and two sliding blocks that are slidably disposed on the second rail, that are respectively adjacent to the rear ends of the pushing rods, and that are located between the actuation operation buttons in the first direction, each of the sliding blocks having a first block surface that faces the first rod surface of the respective one of the pushing rods, the first block surfaces of the sliding blocks facing each other and a distance therebetween increasing forwardly, when the actuation operation buttons are pushed toward each other in the first direction by external forces, the sliding blocks being respectively pushed by the actuation operation buttons, sliding toward each other in the first direction, and respectively pushing the first rod surfaces of the pushing rods via the first block surfaces thereof such that the pushing rods are moved forwardly and that the actuation subunit are converted from the initial state to the actuation state.

12. The suturing device as claimed in claim 11, wherein the actuation subunit further includes a first resilient member that is disposed on the casing and that abuts against front sides of the pushing sections of the pushing rods, and a second resilient member that is located between the sliding blocks, when the actuation subunit is in the actuation state, the second resilient member being resiliently compressed by the sliding blocks, and the pushing sections of the pushing rods pushing the first resilient member forwardly such that the first resilient member is resiliently stretched, when the actuation operation buttons are free from the external forces, the first resilient member exerting a first restoring force on each of the pushing rods such that the pushing rods slide rearwardly, and the second resilient member exerting a second restoring force on each of the sliding blocks such that the sliding blocks slide away from each other and respectively urge the actuation operation buttons to move away from each other, thereby converting the actuation subunit from the actuation state to the initial state.

13. The suturing device as claimed in claim 10, wherein the holding subunit includes an operation rod that is movably disposed on the casing, and a holding resilient member that is located between the operation rod and the casing, the operation rod having a connecting section that is located at a front end thereof, an operation section that is located at a rear end thereof, and a fulcrum section that is rotatably mounted to the casing and that is located between the connecting section and the operation section, the operation section of the operation rod including a holding operation button that is exposed from the casing, the holding portion of the holding subunit including a first holding body that is connected to the front end of the casing, and a second holding body that is connected to the connecting section of the operation rod and that is located above the first holding body in an up-down direction orthogonal to the first direction and the front-rear direction, the holding resilient member resiliently biasing the operation section upwardly such that the second holding body is urged to move downwardly toward the first holding body and cooperate with the first holding body to hold the suturing unit, when the operation section is pressed downwardly, the second holding body being urged to move upwardly away from the first holding body such that the suturing unit is released from the first holding body and the second holding body.

14. The suturing device as claimed in claim 13, wherein each of the first holding body and the second holding body of the holding portion is formed with at least one through hole, the pushing sections of the pushing rods passing through the at least one through hole of each of the first holding body and the second holding body when the pushing rods move forwardly.

15. The suturing device as claimed in claim 10, wherein each of the pushing rods further has a coupling protrusion section that is located at a rear end thereof, the coupling protrusion sections of the pushing rods protruding away from each other in the first direction, the casing further including two operation rails each of which extends in the front-rear direction, the actuation subunit further including two actuation operation buttons that are respectively and slidably disposed on the operation rails of the casing, that are respectively adjacent to the rear ends of the pushing rods, and that are exposed from the casing, each of the actuation operation buttons having a coupling groove section that is coupled to the coupling protrusion section of the respective one of the pushing rods so that when the actuation operation buttons are pushed to slide forwardly by external forces, the pushing rods are urged to move forwardly and the actuation subunit are converted from the initial state to the actuation state.

16. The suturing device as claimed in claim 15, wherein each of the actuation operation buttons further has an operation surface that is exposed from the casing, the operation surfaces of the actuation operation buttons facing away from each other in the first direction and a distance thereof increasing forwardly.

17. The suturing device as claimed in claim 15, wherein the actuation subunit further includes two button resilient members each of which is located between the casing and a respective one of the actuation operation buttons, when the actuation operation buttons are pushed to slide forwardly by the external forces, the button resilient members being respectively and resiliently compressed by the actuation operation buttons, when the actuation operation buttons are free from the external forces, each of the button resilient members exerting a button restoring force to the respective one of the actuation operation buttons to urge the respective one of the actuation operation buttons to slide rearwardly such that the pushing rods are urged to move rearwardly and the actuation subunit are converted from the actuation state to the initial state.

18. The suturing device as claimed in claim 10, wherein the actuation unit further includes a video subunit that includes at least one shooting camera disposed on the casing and facing forwardly so that the at least one shooting camera is operable to record movements of the suturing unit.

19. The suturing device as claimed in claim 18, wherein the video subunit further includes an image processing circuit module that is disposed in the casing and that is electrically coupled to the at least one shooting camera, and at least one camera switch that is electrically coupled to the image processing circuit module and that is operable to control the at least one shooting camera.
